(19) United States
(12) Patent Application Publication
(10) Pub. No.: US 2003/0055357 A1
(43) Pub. Date: Mar. 20, 2003

(54) Method and apparatus for determining electrical parameters of a body

(76) Inventor: Razvan Rentea, Chicago, IL (US)

Correspondence Address: Brinks Hofer Gilson & Lione, P.O. Box 10395, Chicago, IL, 60610, US

(21) Appl. No.: 09/957,362

(22) Filed: Sep. 20, 2001

(51) Int. Cl. A61B 5/04; A61B 5/05; A61H 39/02

(52) U.S. Cl. 600/547; 600/372; 600/548

(57) ABSTRACT

An apparatus for measuring an electrical property of human skin, the apparatus includes a first metallic electrode and an aluminum electrode. The aluminum electrode is made of an aluminum material. The first metallic electrode is made of another conductor such as copper, brass, silver chloride, etc. The apparatus and the electrodes provide improved reliability convenience when measuring skin properties.

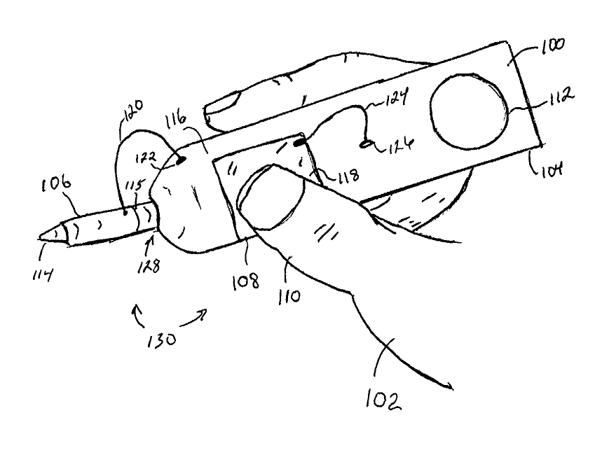

### BACKGROUND

The present invention relates generally to detection of electromagnetic energy or electric current in a human body. More particularly, the present invention relates to materials used in the sensing tips of a device used for determination of electrical potentials and body own electrical currents flowing between an acupuncture point and a remote point on the skin of the body.

Electrical measurements have been made on the skin for over one hundred years. Recent research has shown that the acupuncture points are characterized by lower electrical resistance and higher conductivity than surrounding skin. This is a body phenomenon that may be readily measured as an indicator of the general health and well being of the patient. This phenomenon may also be used to accurately locate one or more acupuncture points.

Therapeutic equipment has been developed to characterize and exploit the resistance properties of the acupuncture points. Such devices have typically been galvanic resistance measurement devices that measure the electrical resistance of the skin upon application of an electrical current. An electrical potential on the order of hundreds of millivolts (mV) and a current on the order of several milli-amperes (mA) are applied to perform the measurement. One particular device or family of devices that has been developed is referred to as Electro Acupuncture According to Voll (EAV) devices. These devices look specifically at the electrical activity of acupuncture points. However, most EAV devices rely on the same principle of galvanic skin resistance.

Recently, a new device was described that operated on the principle of measuring the actual electrical flow between the acupuncture point and a second point located distally on the skin. Such devices are disclosed in German patent publications DE 30 48 358, DE 197 17337, DE 197 17766, DE U 299 02 216. One objection to the galvanic skin resistance measurement technique was the disturbance to skin tissues and biological systems created by application of the electrical voltage and current for performing the electrical measurement. The improved device which measures actual electrical current flow beneficially reduces the disturbance introduced to the biological system by substantially eliminating this voltage and current.

This device, while producing good results, has been less than fully convenient to use. One problem lies in the sensing electrodes which have been used to date, particularly the materials used to construct the electrodes. The industry standard that has developed is silver or silver chloride electrodes, which have permitted measurement of body own electrical currents on the order of 800-900 nA. Silver chloride electrodes are believed to work well because of the presence of the chloride ions in the electrode and in the sweat present on the skin surface which allows relatively good conductive flow of electrical current. Recently, electrodes made of silica have been used for the sensing electrodes. However, they permit measurement of body own electrical currents on the order of 25 nano-amperes (nA).

The silver chloride electrodes also have been inconvenient to use. To ensure a good electrical contact between the electrode and the skin, the silver chloride and other prior art electrodes have required application of a conductive paste or gel to the skin. This can be messy and inconvenient for the patient and adds to the time and expense required for the measurement process. Further, the measurement process is relatively slow using the silver chloride electrodes.

Accordingly, there is a need in the art for an improved method and apparatus for measuring electrical parameters of the skin which provides reliable, accurate measurement and user convenience.

### BRIEF SUMMARY

By way of introduction, an improved apparatus for measuring electrical parameters of the skin is provided by an apparatus which uses a first metallic electrode in conjunction with an aluminum electrode. In one embodiment, the first metallic electrode includes copper, brass or other cuprous metal. In another embodiment, the first metallic electrode includes silver or silver chloride. In yet another embodiment, both electrodes include aluminum. The aluminum electrode may be made of any suitable alloy of aluminum and in one embodiment is characterized by having a higher contact resistance with the human skin than the first metallic electrode.

It has been found that a particular film thickness of aluminum or aluminum alloy is required to produce good results. Improved results are produced with a film of aluminum less than about 0.25 millimeters (mm) thick and best results have been produced with a film of aluminum about 100 mm thick. This film may be a piece of aluminum foil or tape or may be formed on a substrate, for example by depositing aluminum atoms on the substrate to a controlled thickness.

A biofeedback device such as an EAV device may be produced using such electrodes. Further, the electrodes may be produced and supplied as replacement electrodes for a biofeedback device such as an EAV device.

An improved method for measuring electrical activity of the skin is provided by directly contacting the skin with a first metallic electrode, such as an electrode made of copper, brass or other copper alloy, silver chloride or aluminum, while also directly contacting the skin with an second electrode which includes a substantial portion of aluminum. An electrical signal is then detected between the first metallic electrode and the second electrode.

The foregoing introduction of the preferred embodiments has been provided only by way of introduction nothing in this section should be taken as a limitation of the following claims which define the scope of the invention.

### BRIEF DESCRIPTION OF THE DRAWING

The embodiments of the invention, together with further objects and advantages thereof, may best be understood by making reference to the accompanying drawing, the sole FIGURE of which is a biofeedback device grasped by a human hand for measurement of electrical properties of skin.

### DETAILED DESCRIPTION

Referring now to the drawing, it shows one exemplary embodiment of a biofeedback device 100 grasped by a human hand 102. The biofeedback device 100 is configured to measure an electrical property of human skin and includes a housing 104, a first electrode 106 and a second electrode 108. In the illustrated embodiment, the biofeedback device 100 is graspable by a human hand such as the human hand 102 so that the first electrode 106 may be placed in direct electrical contact with the skin of the human at a desired location and the second electrode 108 is in direct electrical contact with a digit or finger of the human hand 102 such as thumb 110. In other embodiments, the biofeedback device 100 may be located remotely from one or more of the electrodes 106, 108 but remain in electrical contact with the electrodes 106, 108.

In the illustrated embodiment, the biofeedback device 100 is an Electro Acupuncture According to Voll (EAV) device which measures electrical activity associated with acupuncture points. In particular, the biofeedback device 100 is configured to measure an electrical current at an acupuncture point and produce an indication. Accordingly, the biofeedback device 100 in a typical embodiment includes electrical circuitry contained within the housing 104, a battery also contained within the housing 104 and a user interface 112 for providing the indication and for otherwise providing user control of operation of the biofeedback device 100. The user interface 112 may include a digital display, a meter or any other suitable device for providing operating information including information about electrical activity of the skin measured by the biofeedback device 100. The electrical circuitry contained in the housing may be any suitable device or combination of devices for measuring electrical properties of the skin, such as Electro Acupuncture According to Voll.

The electrodes 106, 108 are preferably electrically conductive, at least in part. Each electrode 106, 108 includes a skin-contacting portion configured for direct engagement with the skin of a patient. Thus, the first electrode 106 includes a skin-contacting portion in the form of a tapered tip 114 to contact an acupuncture point of a patient's skin. The second electrode 108 is disposed on a side 116 of the housing 104 and positioned so that the second electrode 108 presents a large, flat skin-contacting surface 118 which directly engages a finger of the patient's hand. In the illustrated embodiment, the thumb 110 of the hand 102 is engaged by the second electrode 108, but the second electrode 108 could alternatively be positioned or shaped in any suitable configuration to engage any other portion of the hand or other skin of the patient. The remainder of the electrode other than the skin-contacting portion may be formed of any suitable material, including even an insulator. For example, the shaft 115 of the first electrode 106 other than the tip 114 may be formed of a plastic material to provide advantages such as sterility, replaceability, mechanical support, and electrical isolation.

The conducting electrodes 106, 108 are also in electrical contact with electrical circuitry contained within the housing 104 for sensing an electrical signal at at least one of the first electrode 106 and the second electrode 108. In the illustrated embodiment, a wire 120 extends from the first electrode 108 through an aperture 122 in the housing 104 to electrically contact the electrical circuitry in the housing 104. Similarly, a wire 124 extends from the second electrode 108 through an aperture 126 to electrically contact the electrical circuitry in the housing 104. Preferably, the wires 120, 124 are highly conductive to provide an accurate indication of an electrical parameter for measurement by the biofeedback device 100. Copper wire has been found to work well. In place, of the wires 120, 124, the electrical contact may alternatively be made directly to the electrical circuitry. For example, the probe formed by the first electrode 106 may be inserted into an end aperture 128 in the housing 104 so that a conductive end of the probe engages a conductive post, plate or annulus within the housing 104 which is in electrical contact with the electrical circuitry 104. Similarly, the second electrode 108 may engage a conductive post or plate under the electrode 108.

In accordance with one embodiment, at least one of the electrodes 106, 108 is an aluminum electrode. That is, at least one of the electrodes includes a skin-contacting portion which is made from or includes aluminum, an alloy or chemical compound of aluminum, either fully or in substantial part. In experiments with a device similar to the illustrated biofeedback device 100, it has been found that a first electrode made of copper or brass or other cuprous metal, in combination with a second or aluminum electrode made of aluminum foil, provides excellent results. Body own electrical currents in excess of 1600 nano-Amperes (nA) have been measured with this device. Other embodiments may be substituted and provide excellent results as well, as will be describe herein.

In one alternative embodiment, one of the electrodes 106, 108 is an aluminum electrode and the other electrode includes silver chloride. In this manner, the aluminum electrode may be used in combination with a silver chloride electrode, which is widely available. In another alternative embodiment, the other electrode is also aluminum or a conductor made with a substantial portion of aluminum. Other equivalent metals, alloys or combinations of materials may be substituted as well. The relative positioning and location of the aluminum electrode and the first electrode may be varied also. For example, in the illustrated embodiment, the first electrode 106 may be formed of aluminum material and the second electrode 108 may be formed of another conductor such as a cuprous metal like brass, silver chloride or even aluminum. By cuprous metal, it is meant a metal or alloy or other conductive material containing copper in substantial portion.

It has been found that the configuration and dimensions of the aluminum electrode can have a strong effect on performance of the biofeedback device. In one embodiment, the aluminum electrode is formed of a metallic layer having a predetermined thickness. Preferably, the thickness of the metallic layer is less than about 0.25 millimeters (mm). It has been found that when aluminum is used, thicker metallic layers do not work well or at all. Most preferably, the thickness of the metallic layer is about 0.1 mm.

In one embodiment, the metallic layer is an aluminum film. The film may be formed using aluminum foil adhered to a substrate. Alternatively, the film may be formed by depositing an aluminum film on a substrate, for example using a chemical vapor deposition or sputtering process. The substrate preferably provides mechanical support and protection for the aluminum foil or film. In a simple and inexpensive embodiment, a piece of aluminum foil having a suitable thickness is taped directly to the skin of the patient or, as shown in the illustrated embodiment, to the housing of the biofeedback device 100.

It is a particular advantage of the electrodes described herein that they are configured for direct contact with the skin of a patient. No conductive past or gel is needed. In the illustrated embodiment, the skin of the thumb 110 is contacted when the device 100 is grasped by the patient. The tip 114 is configured for directly contacting the skin at a remote location of the patient. More particularly, the first electrode is pointed at the tip 114 to contact an acupuncture point of the patient's skin. The patient may grasp the device 100 as shown and place the tip 114 against selected acupuncture points of the patient's body. This embodiment is thus well adapted for measuring Electro Acupuncture according to Voll associated with acupuncture points of the patient's skin.

It is believed that the aluminum electrode has a higher contact resistance with the skin than electrodes of other materials, such as cuprous metals or silver chloride. This higher contact resistance suggests that an aluminum electrode would be a poor choice for measuring electrical properties of the skin, since the higher contact resistance could interfere with reliable and accurate measurements of small electrical currents and potentials. However, after much experimentation with different materials and mechanical properties, the opposite has been found to be true. The use of an aluminum electrode is an important feature of the embodiments disclosed herein.

In another embodiment, the first electrode 106 and the second electrode 108 may be combined as an electrode set. In this embodiment, an electrode set 130 for a biofeedback device such as the biofeedback device 100 includes a first metallic electrode such as first electrode 106 formed for electrical contact with a measuring portion such as the internal electrical circuitry of the biofeedback device and an aluminum electrode such as second electrode 108 formed for electrical contact with the measuring portion of the biofeedback device. The respective electrodes should have the electrical and mechanical properties as described herein. The electrode set 130 may in this manner be provided as replacement electrodes for use with the biofeedback device if the original electrodes become damaged, worn, dirty or otherwise need replacement.

In a further embodiment, the shaft 115 of the first electrode 106 may be keyed to match the end aperture 128 in the housing 104 so that only approved electrode sets may be used with the biofeedback device. That is, the end of the shaft which is inserted in the end aperture 128 in the housing 104 may have a particular outer shape which mechanically engages the end aperture 128 which a matching particular inner shape. Alternatively, one or both electrodes 106, 108 may provide a predetermined electrical response which is required for operation of the biofeedback device. These features may provide advantages such as ensuring that only high-quality conforming electrodes are used with the biofeedback device.

From the foregoing, it can be seen that the present invention provides an improved method and apparatus for measuring electrical parameters of the skin which provides reliable, accurate measurement and user convenience. When used in conjunction with an EAV device, excellent measurement results have been produced, measuring currents in excess of 1600 nA, a value not previously achieved. Further, no conductive gel or other material is necessary when using the disclosed embodiments. The disclosed electrodes directly contact the skin and provide the noted excellent results. The materials found to work well are inexpensive and the disclosed electrodes may be manufactured inexpensively and with high quality.

While a particular embodiment of the present invention has been shown and described, modifications may be made. It is therefore intended in the appended claims to cover all such changes and modifications which fall within the true spirit and scope of the invention.

1. Apparatus for measuring an electrical property of human skin, the apparatus comprising:
a first metallic electrode; and
an aluminum electrode.

2. The apparatus of claim 1 wherein the aluminum electrode comprises a metallic layer having a thickness less than 0.25 millimeters.

3. The apparatus of claim 1 wherein the aluminum electrode comprises a metallic layer having a thickness of about 0.1 millimeters.

4. The apparatus of claim 2 wherein the aluminum electrode comprises an aluminum layer deposited on a substrate.

5. The apparatus of claim 1 wherein the first metallic electrode comprises a cuprous metal.

6. The apparatus of claim 1 wherein the first metallic electrode comprises one of copper and brass.

7. The apparatus of claim 1 wherein the first metallic electrode comprises silver chloride.

8. The apparatus of claim 1 wherein the first metallic electrode comprises aluminum.

9. The apparatus of claim 1 wherein the first metallic electrode and the aluminum electrode are configured for direct engagement with the skin of a patient.

10. The apparatus of claim 1 wherein the aluminum electrode has a higher contact resistance with the human skin than the first metallic electrode.

11. A method for measuring electrical activity of skin, the method comprising:
directly contacting the skin with a first metallic electrode;
directly contacting the skin with a second metallic electrode including a substantial portion of aluminum; and
detecting an electrical signal between the first metallic electrode and the second metallic electrode.

12. The method of claim 11 wherein directly contacting the skin with the first metallic electrode comprises:
contacting the skin with a cuprous portion of a metal electrode.

13. The method of claim 11 wherein directly contacting the skin with the second metallic electrode comprises:
contacting the skin with an aluminum portion of an electrode.

14. The method of claim 11 wherein directly contacting the skin with the second metallic electrode comprises:
contacting the skin with an aluminum alloy portion of an electrode.

15. The method of claim 11 further comprising:
applying an electric potential across the first metallic electrode and the second metallic electrode; and
measuring an electrical parameter of the skin.

16. The method of claim 15 further comprising:
contacting the skin of a patient's first hand with the first metallic electrode; and
contacting the skin of the patient's second hand with the second metallic electrode.

17. The method of claim 16 further comprising:
contacting the skin of the patient's first hand at a first acupuncture point; and
contacting the skin of the patient's second hand at a second acupuncture point.

18. A biofeedback device comprising:
a first metallic electrode configured for direct electrical contact with a first predetermined portion of a patient's skin;
a second metallic electrode substantially comprising aluminum and configured for direct electrical contact with a second portion of the patient's skin;
electrical circuitry for sensing an electrical signal at at least one of the first metallic electrode and the second metallic electrode; and
user interface means for providing information about an electrical property of the patient's skin.

19. The biofeedback device of claim 18 wherein the second metallic electrode comprises an aluminum film having a thickness less than about 0.25 millimeters.

20. The biofeedback device of claim 19 wherein the second metallic electrode comprises an aluminum film having a thickness of about 0.1 millimeters.

21. The biofeedback device of claim 19 wherein the second metallic electrode comprises aluminum tape positioned on a portion of the biofeedback device for engagement by a finger of the patient when the patient grasps the biofeedback device.

22. The biofeedback device of claim 18 wherein the first metallic electrode comprises:
a skin-contacting portion formed at least in part of one or more of silver chloride, copper and brass.

23. The biofeedback device of claim 22 wherein the skin-contacting portion of the first metallic electrode is pointed to contact an acupuncture point of the patient's skin.

24. The biofeedback device of claim 18 wherein the second metallic electrode comprises an aluminum film having a thickness less than about 0.25 millimeters.

25. The biofeedback device of claim 18 comprising apparatus for measuring Electro Acupuncture according to Voll associated with acupuncture points of the patient's skin.

26. An electrode set for a biofeedback device comprising:
a first metallic electrode formed for electrical contact with a measuring portion of the biofeedback device; and
an aluminum electrode formed for electrical contact with the measuring portion of the biofeedback device.

27. The electrode set of claim 26 wherein the first metallic electrode comprises a cuprous metal.

28. The electrode set of claim 26 wherein the first metallic electrode comprises silver chloride.

29. The electrode set of claim 26 wherein the first metallic electrode comprises a skin contacting portion for direct electrical contact with skin.

30. The electrode set of claim 26 wherein the aluminum electrode comprises a skin contacting portion of aluminum-containing metal for direct electrical contact with skin.

31. The electrode set of claim 30 wherein the aluminum electrode comprises an aluminum file having a thickness less than about 0.25 millimeters.

32. The electrode set of claim 31 wherein the aluminum electrode comprises an aluminum file having a thickness about 0.1 millimeters.
